(19) United States
(12) Patent Application Publication
(10) Pub. No.: US 2015/0273089 A1
(43) Pub. Date: Oct. 1, 2015

(54) METHOD OF TREATING CANCER

(71) Applicant: Bruce N. GRAY, (US)

(72) Inventor: Bruce N. Gray, Wahroonga (AU)

(21) Appl. No.: 14/428,613

(22) PCT Filed: Sep. 17, 2013
(86) PCT No.: PCT/AU2013/001059

§ 371 Date: Mar. 16, 2015

(30) Foreign Application Priority Data
Sep. 17, 2012 (AU) 2012904066

(51) Int. Cl. A61K 51/12; A61K 51/02; A61K 51/06

(52) U.S. Cl. CPC A61K 51/1251; A61K 51/06; A61K 51/025

(57) ABSTRACT

The present invention relates to small particles comprising a radionuclide and in particular to small particles comprising a radionuclide for implantation in organs or tissues or tumours of subjects. Embodiments of the invention have been particularly developed for embolisation into the arterial system using a technique known as radioembolisation or Selective Internal Radiation Therapy (SIRT) and will be described hereinafter with reference to this application. However, it will be appreciated that the invention is not limited to this particular field of use. The small particles are preferably radioactive microspheres comprising a matrix and a radionuclide stably attached. These microspheres have a diameter ranging from 5 to 45 μm and the radionuclide has a specific activity ranging from 100 to 2000 Bq per microsphere.

### FIELD OF THE INVENTION

The present invention relates to small particles comprising a radionuclide and in particular to small particles comprising a radionuclide for implantation in organs, tissues or tumours of subjects. Embodiments of the invention have been particularly developed for embolisation into the arterial system using a technique known as radioembolisation or Selective Internal Radiation Therapy (SIRT) and will be described hereinafter with reference to this application. However, it will be appreciated that the invention is not limited to this particular field of use.

### BACKGROUND

Any discussion of the background art throughout the specification should in no way be considered as an admission that such art is widely known or forms part of common general knowledge in the field.

SIRT is usually applied in order to implant radioactive microspheres into a target such as an organ, tissue or tumour so as to deliver ionising radiation to that target resulting in damage or death of that target organ, tissue or tumour. As indicated, the tissues may be normal body organs or tissues, or benign or malignant tissues (collectively referred to as tumours).

Previously described radioactive microspheres for therapeutic application typically comprise a matrix material that can act as a carrier for a radionuclide material which emits ionising radiation. Such radioactive microspheres have been utilised to deliver radiation to targets such as organs, tissues or tumours.

In particular, it has previously, been shown that a number of beta radiation emitting radionuclides, such as Phosphorus-32, Holmium-166 or Yttrium-90, could be attached to matrix microspheres such as polymeric resin or glass microspheres and that the resulting radioactive microspheres (particulate material) could be injected into the blood stream of a cancer patient with therapeutic effect.

In the known methods for Selective Internal Radiation Therapy (SIRT) radioactive microspheres are generally delivered via the arterial blood supply of the target tissue or tumour. To this end, generally, a catheter is guided to the branch of the blood vessel that feeds the target tissue or tumour to infuse the microspheres into the circulation. The radioactive microspheres become trapped in the capillary beds of target tissue or tumour providing for the selective delivery of a dose of radiation to the target tissue or tumour.

An alternate and less common method of delivering radioactive microspheres is by direct injection into the tumour.

In the known SIRT methods of treating liver cancer in humans, radioactive microspheres are usually introduced into the arterial blood supply of either the whole liver, a section of the liver, or into the arterial blood supply of that part of the liver containing the tumour that is to be treated, by injection of the radioactive microspheres into the hepatic artery, the portal vein, or a branch of either of these vessels.

There are currently two commercially-available products readily available for SIRT to treat liver cancer, namely, TheraSphere® (MDS Nordion, Inc.), and SIR-Spheres® (SIRTeX® Medical Ltd.). Both products are Yttrium-90 labelled microspheres: TheraSpheres® being glass microspheres having a diameter of 25±10 μm; and SIR-Spheres® being resin-based microspheres that having a diameter of 32±2.5 μm. As indicated above, during SIRT the radioactive microspheres become lodged in the pre-capillary or capillary network of the target tissue or tumour. However, it is a concern with both of these products that a proportion of the injected radioactive microspheres travels to healthy, non-target tissues, e.g. to the lungs, pancreas, gallbladder, stomach and/or duodenum where the exposure to radiation causes undesired side effects.

The FDA approved specifications for SIR-Spheres® are that 30-60×10⁶ microspheres are used for each 3 GBq Of activity at the approximate time of treatment—which equates to a specific activity of 50-100 Bq/sphere. As such, according to Vente et al. 2009, in order to deliver the standard does of 3 GBq of ⁹⁰Y to a patient, approximately 50×10⁶ SIR-Spheres® have to be administered. However, in clinical practice, physicians are regularly unable to deliver the desired radiation dose to the patient because the physiological limitations of the vascular capacity of the target tissue or tumour do not allow for the delivery of the pre-determined amount of SIR-Spheres® required to deliver the desired dose. As a result, SIRT using SIR-Spheres® has been frequently associated with undesirable reflux of SIR-Spheres® into extra-hepatic organs blockage of the arterial tree resulting in serious adverse events following the treatment with SIR-Spheres® (Vente et al. 2009 Yttrium-90 microsphere radioembolization for the treatment of liver malignancies: a structured meta-analysis. Eur Radiol. 2009 April; 19(4):951-9).

To mitigate such potentially serious complications, radiologists administering SIR-Spheres® often undertake real-time visualisation of the arterial vasculature of the target tumour and non-target tissues carefully monitoring whether any reflux of infused microspheres occurs such that administration can be ceased if any signs of reflux is observed.

An alternative strategy to avoid the above-noted problem of microsphere reflux is the use of a specialised arterial catheter designed to stop microsphere reflux (for example, see Surefire Infusion System ST/LT by Surefire Medical Inc., Westminster, Colo. 80031, USA).

In contrast to Sirtex Medical Ltd.'s SIR-Spheres®, MDS Nordion Inc.'s TheraSpheres® are ⁹⁰Y-labelled glass microspheres with a much higher specific activity of approximately 2,500 Bq/sphere (Vente et al. 2009). As a result, only approximately 4×10⁶ TheraSpheres® (i.e. less than one tenth of the number of SIR-Spheres®) need to be administered to deliver the standard TheraSpheres® dose of 5 GBq ⁹⁰Y and as such, reflux resulting from arterial blockage does not pose a major problem during SIRT using TheraSpheres®. Notwithstanding, Vente et al. 2009 reported that SIR-Spheres® were significantly more effective in treating liver cancer than TheraSpheres® (89% vs. 78% (p=0.02)) and speculated that the low number of microspheres infused may be a disadvantage when targeting a tumor type that is often diffusely spread throughout the liver at the time of diagnosis.

The above are well-recognised problems of the current products available for SIRT to treat liver cancer and while some SIRT liver cancer patients have suffered from radiation damage to non-target tissues, both above-mentioned products are widely used in practice. In fact, both products have proven to be commercially very successful, and having been largely associated with therapeutic success, have become the “gold-standard” for SIRT of liver cancer.

### SUMMARY OF THE INVENTION

When using radioembolisation to treat a tumour, it is desirable to selectively deliver high doses of ionising radiation to the tumour but only low radiation doses to the surrounding non-target organs or tissues. The selective high radiation dose will result in damage or death of the tumour while sparing the normal tissues from excessive radiation injury (i.e. Selective Internal Radiation Therapy (SIRT)).

The liver, for example, is supplied by blood from both the hepatic artery (including one or more accessory hepatic arteries) and the portal vein. In contrast, tumours within the liver receive the majority of their blood supply only from the arterial supply to the liver and not the portal vein. Therefore, any radioactive microspheres injected into the main hepatic artery, accessory hepatic arteries, segmental hepatic arteries of sub-segmental hepatic arteries will preferentially be directed to the tumour(s) within the liver in greater concentration that in the normal liver tissue. This in turn will result in preferential irradiation of the tumour compared to the normal liver tissue.

Notwithstanding, if radioactive microspheres are delivered into the blood stream of any target tissue or tumour, the amount of radioactive microspheres that can be selectively delivered is generally determined by the capacity of the blood vessel network to accommodate those radioactive microspheres.

In addition, the amount and effectiveness of radiation selectively delivered to the target tissue or tumour will depend on a number of other factors, including the spatial distribution of the radioactive microspheres within the target tissue or tumour, the type of radionuclide used, the amount of radionuclide used and the specific activity of the radionuclide used.

When radioactive microspheres are delivered by injection into the arterial blood supply of the target tissue or tumour, as discussed above, the radioactive microspheres become lodged in the pre-capillary or capillary network of the target tissue or tumour. The resulting spatial distribution of radioactive microspheres in the target tumour compared to the surrounding non-target tissues will determine the success of the treatment, i.e. the effectiveness of delivering a tumouricidal dose to the tumour while, at the same time, sparing the non-target tissue from excessive and damaging exposure to radiation.

The spatial distribution of radioactive microspheres in the target tumour compared to the surrounding non-target tissues depends on many factors, including the number, size, shape, density and flow characteristics of the radioactive microspheres themselves as well as on the relative blood flow rate and blood flow volume of the target tumour compared to the surrounding non-target tissues and on the relative capacity of the blood vessels in the tumour and non-target tissue compartments.

However, and as indicated above, it would appear that neither of the currently available radioactive microspheres have the optimal specific activity for SIRT. For example, SIR-Spheres® have a specific activity ranging from approximately 30-100 Bq per sphere and are provided in a vial containing a predetermined amount of total radioactivity (usually between 2 and 3 GBq). The total dose does not take the varying specific activity per microsphere into account. As such, the total number of radioactive microspheres per vial (i.e. the number of radioactive microspheres required to be administered to deliver the predetermined radiation dose) varies significantly in practice. Accordingly, and while still useful in SIRT, it is recognised that the uncontrolled variability in relation to the total number of microspheres and the specific activity of radioactive microspheres to be administered to each individual patient may be the cause for the complications and undesirable side-effects seen in SIRT.

Accordingly, there is a need in the art for improved radioactive microspheres and methods of their manufacture as well as their use, where the number of radioactive microspheres to be administered and their specific radioactivity is calculated based on the characteristics of the target tissue or tumour to be treated such that the SIRT effect can be optimised by delivering the highest possible radiation dose to the target tissue or tumour while minimising any deleterious radiation effects on non-target tissues.

It Is an object of the present invention to overcome or amelio to at least one of the disadvantages of the prior art, or to provide a useful alternative.

Fox et al. (1991, Dose distribution following selective internal radiation therapy. Int J Radiat Oncol Biol Phys.), Campbell et al. (2000, Analysis of the distribution of intraarterial microspheres in human liver following hepatic yttrium-90 microsphere therapy. Phys Med Biol. April; 45(4):1023-33; and 2001, Tumour dosimetry in human liver following hepatic yttrium-90 microsphere therapy. Phys Med Biol. February; 46(2):48-98.) and others have shown that radioactive microspheres infused via the hepatic artery during SIRT of liver cancer distribute non-uniformly throughout the liver and preferentially lodge in the tumour vasculature, whilst sparing normal, non-target tissues. Accordingly, the resulting radiation dose distributions were also non-uniform, with a larger dose being delivered to the tumour as compared to normal, non-target tissues.

Specifically, it was noted that microspheres injected into the artery of tumour bearing liver accumulate in clusters rather than as individual microspheres in both the vasculature of the target tumour as well as in normal, non-target liver parenchyma. Further, it was noted the clusters accumulate in the tumour vasculature and appear approximately five times closer to each other in the tumour than in the normal liver parenchyma.

Importantly, the present inventor noted that this heterogeneous, clustered microsphere distribution can be utilised to maximise the beneficial, therapeutic effects of SIRT while minimising the undesirable side-effects seen with presently used microspheres.

in particular, the present inventor has surprisingly found that the heterogeneous, clustered microsphere distribution in the tumour's vasculature leads to the amplification of the radiation dose delivered by each microsphere, due to the overlap of radiation dose emitted by each sphere.

In testing the converse, the inventor has found that increasing the specific activity per microsphere and proportionally lowering the number of microspheres to be administered has only a minimal effect on the dose distribution throughout the peripheral tumour vasculature and still provides therapeutically effective dosimetry. This effect is attributed to the amplification due to the overlapping emission of radiation dose by spheres within each cluster but also in neighbouring clusters. However, as fewer microspheres are delivered in total, the clusters seen in liver parenchyma are populated with fewer microspheres and, due to their relative isolation from other clusters, the radiation exposure of non-target liver tissue is significantly reduced.

Furthermore, raising the specific activity per microsphere in accordance with the present invention allows for a significant reduction in the number of microspheres to be infused to deliver a predetermined dose of radiation, thereby significantly reducing the risk of undesirable microsphere reflux without compromising therapeutically effective distribution throughout the peripheral tumour vasculature.

In light of the observations made by Vente et al. 2009 that infusion of a lower number of TheraSpheres® having a high specific activity appears to lead to undesirable dose distribution, potentially affecting the effectiveness of the TheraSpheres® in SIRT, it was surprisingly found that reducing the number of radioactive microspheres while proportionally increasing the specific activity per microsphere (even by a factor of 10) does not have a significant effect on the radiation dose delivered. Calculated dose distributions suggest that the tumour periphery receives a therapeutic dose for all of the embodiments of the present invention tested.

As such, the present inventor has recognised that the above-described undesirable side-effects seen in current SIRT practice constitute avoidable problems which can be mitigated by using the microspheres of the present invention having an optimised specific radiation activity per microsphere and that, as such, the often serious clinical complications arising from current SIRT practice can be minimised.

Accordingly, in a first aspect the present invention relates to a radioactive microsphere comprising a microsphere matrix and a radionuclide stably attached to, or incorporated within, said matrix,

wherein said microsphere has a diameter ranging from about 5 to about 45 μm, and

wherein said radionuclide provides a specific activity to said microsphere, said specific activity ranging from more than 100 Bq to less than 2000 Bq per microsphere.

In some preferred embodiments, the specific activity ranges from about 150 Bq to about 1500 Bq per microsphere. In further preferred embodiments, the specific activity ranges from about 200 Bq to about 1500 Bq per microsphere. Typically, the specific activity ranges from about 200 Bq to about 500 Bq per microsphere. However, in some further preferred embodiments the specific activity ranges from more than 100 Bq to less than 1000 Bq per microsphere. Alternatively, the specific activity ranges from more than 100 Bq to about 500 Bq per microsphere.

Preferably, the radioactive microsphere of the first aspect is for use in Selective Internal Radiation Therapy (SIRT). Preferably, the SIRT is SIRT to treat cancer. Preferably, the cancer is liver cancer. Alternatively, the cancer is colorectal cancer.

Preferably, the radionuclide is an isotope selected from isotopes of Yttrium, Holmium, Samarium, Iodine, Phosphorus, Iridium and Rhenium. Typically, the radionuclide is an isotope of Yttrium, preferably 90-Yttrium (⁹⁰Y).

In some preferred embodiments the microsphere matrix is a polymeric matrix. Preferably, the polymeric matrix is an ion exchange resin comprising partially cross-linked polystyrene, and the ⁹⁰Y is precipitated as a ⁹⁰Y-phosphate salt such that said phosphate salt is stably attached to said matrix by adsorbtion onto the surface of said ion exchange resin.

Alternatively, in some preferred embodiments the microsphere matrix is glass.

Preferably, the microsphere provides for a radioactive dose distribution throughout a target tissue similar to that of polymeric microspheres having a specific activity of about 50 Bq per microsphere and reduces the risk of undesirable side-effects of SIRT resulting from radioactive microsphere reflux.

In a second aspect, the present invention relates to a method of producing a radioactive microsphere comprising the step of combining a microsphere matrix with radionuclide for a time and conditions sufficient to stably attach a specified portion of said radionuclide to said microsphere matrix, or to stably incorporate said specified portion of said radionuclide within said matrix, thereby producing a radioactive microsphere having a diameter ranging from about 5 to about 45 μm, wherein said specified portion of radionuclide provides a specific activity to said microsphere, said specific activity ranging from more than 100 Bq to less than 2000 Bq per microsphere.

In a third aspect, the present invention relates to a radioactive microsphere when produced by the method of the second aspect.

In a fourth aspect, the present invention relates to a method of SIRT comprising administering the microsphere of first or third aspect to a subject in need thereof.

Typically, the subject has cancer and the SIRT targets said cancer. Generally, the cancer is selected from liver and colorectal cancer.

In a fifth aspect, the present invention relates to use of a microsphere of first or third aspect for the manufacture of a medicament for the treatment of cancer.

Typically, the medicament is adapted for use in SIRT. Preferably, the SIRT targets the cancer. Generally, the cancer is selected from liver and colorectal cancer.

Reference throughout this specification to “one embodiment”, “some embodiments” or “an embodiment” means that a particular feature, structure or characteristic described in connection with the embodiment is included in at least one embodiment of the present invention. Thus, appearances of the phrases “in one embodiment”, “in some embodiments” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

As used herein, unless otherwise specified the use of the ordinal adjectives “first”, “second”, “third”, etc., to describe a common object, merely indicate that different instances of like objects are being referred to, and are not intended to imply that the objects so described must be in, a given sequence, either temporally, spatially, in ranking, or in any other manner.

In the context of this specification the following terms are defined as follows:

### “Microsphere Matrix”

As used herein, the term “microsphere matrix” relates to a physiologically inert material which can act as a carrier for the radionuclide emitting the ionising radiation required for SIRT.
“Stably Attached to, or Incorporated within, Said Matrix”
In the context of the present specification the above term means that the radionuclide is attached to the surface of the microsphere matrix, or incorporated within the microsphere matrix, such that less than 0.4%, preferably, less than 0.2% or 0.1%, more preferably less than 0.07%, 0.05%, 0.03% or 0.01% of the radionuclide leaches from the radioactive microspheres under physiological conditions. When the radionuclide is attached to the surface of the microsphere matrix, the surface may be of any confirmation, for example, it may be smooth, undulating, pitted or porous.

### “Specific Activity”

In the context of the present specification, the term “specific activity” is intended to refer to the activity of a particular radionuclide per microsphere.

### “Ion Exchange Resin”

In the context of the present specification “ion exchange resin” refers to a polymeric matrix (normally in the form of microspheres), which are typically porous, providing a high surface area. These microspheres allow for the trapping/binding of ions by ion-exchange. Typically, ion exchange resins are based on cross linked polystyrene where the actual ion exchanging sites are introduced after polymerization. Additionally, in the case of polystyrene, cross linking is introduced via copolymerization of styrene and a few percent of divinylbenzene.

### “Undesirable Side-Effects of SIRT”

In the context of the present specification, the term “undesirable side-effects of SIRT” is meant to include all previously reported side-effects and, without being limited, these are meant to include:
Inadvertent delivery of microspheres to non-target tissues or organs such as the stomach or pancreas causing abdominal pain and nausea, acute pancreatitis or peptic ulceration (stomach ulcer).
Excessive radiation to normal, non-target tissue. In the liver this may result in radiation hepatitis.
Inadvertent delivery of radioactive microspheres to the gall bladder which, in turn, may result in inflammation of the gall bladder.

### “Specified Portion of Said Radionuclide”

In the context of the present specification, the term “specified portion of said radionuclide” is intended to refer to a predetermined amount of radioactivity stably attached to, or incorporated within the microsphere matrix thereby providing the specific activity to the microspheres of the present invention ranging from more than 100 Bq to less than 2000 Bq per microsphere. The predetermined amount of radioactivity to be stably attached to, or incorporated within the microsphere matrix, can be controlled, for example, by adjusting the total amount of radioactivity or by adjusting the amount of microsphere matrix available.

### “Exemplary”

As used herein, the term “exemplary” is used in the sense of providing examples, as opposed to indicating quality. That is, an “exemplary embodiment” is an embodiment provided as an example, as opposed to necessarily being an embodiment of exemplary quality.

### “Comprising”

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

### BRIEF DESCRIPTION OF THE DRAWINGS

Embodiments of the invention will now be described, by way of example only, with reference to the accompanying drawings in which:

FIG. 1A illustrates selected isodose contours for a central tissue section through the tumour-normal tissue boundary of a liver tumour modelled for microspheres of the present invention having a specific activity of 200 Bq per sphere when infused to deliver a 3 GBq dose (i.e. the total number of microspheres infused represents a quarter of the number of SIR-Spheres® required to deliver the equivalent radiation dose at 50 Bq per SIR-Sphere®). The “+” symbol indicates the position of radioactive microspheres having a specific activity of 50 Bq observed in tissue sections and the “0” indicates a quarter of those microspheres which were randomly selected to serve as the positions from which the isodose contours for 200 Bq microspheres have been determined. The dashed line on the left hand side shows the tumour boundary and the line on the right is the edge of the tumour vascular periphery, indicating that microsphere clusters predominantly occur in the tumour periphery.

FIG. 1B shows more detailed isodose contours of the boxed area shown in FIG. 1A.

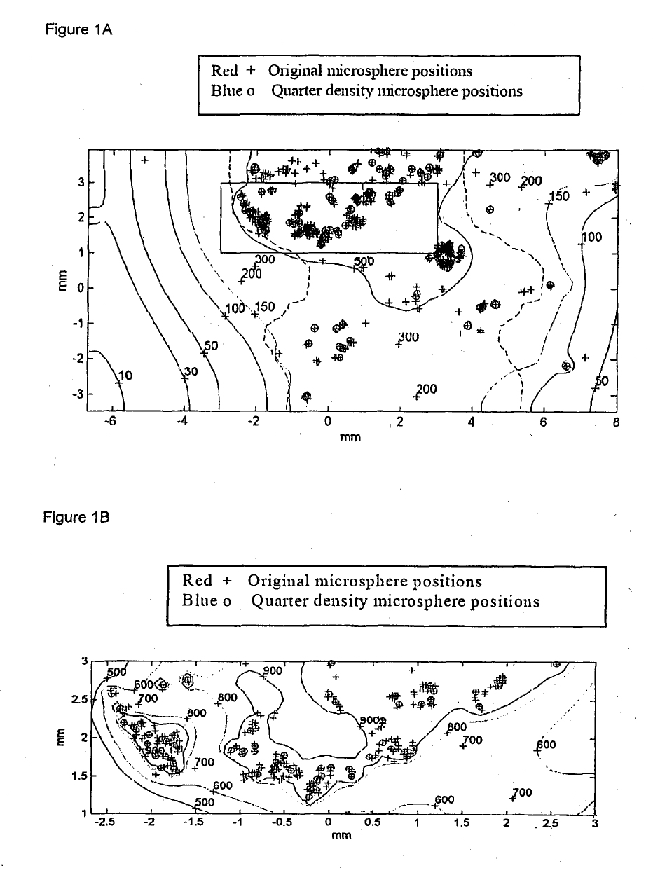

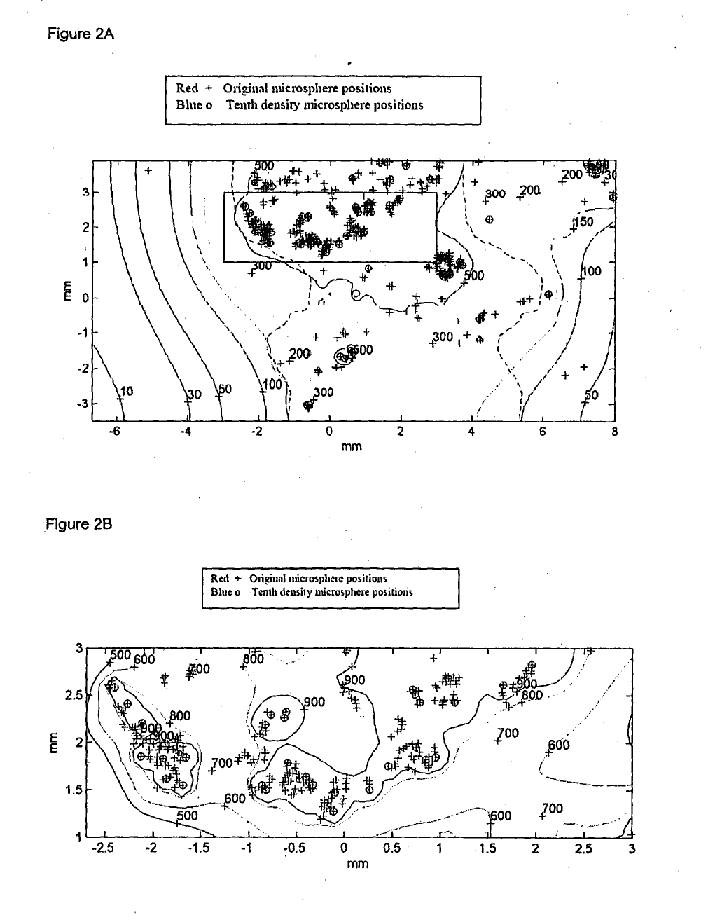

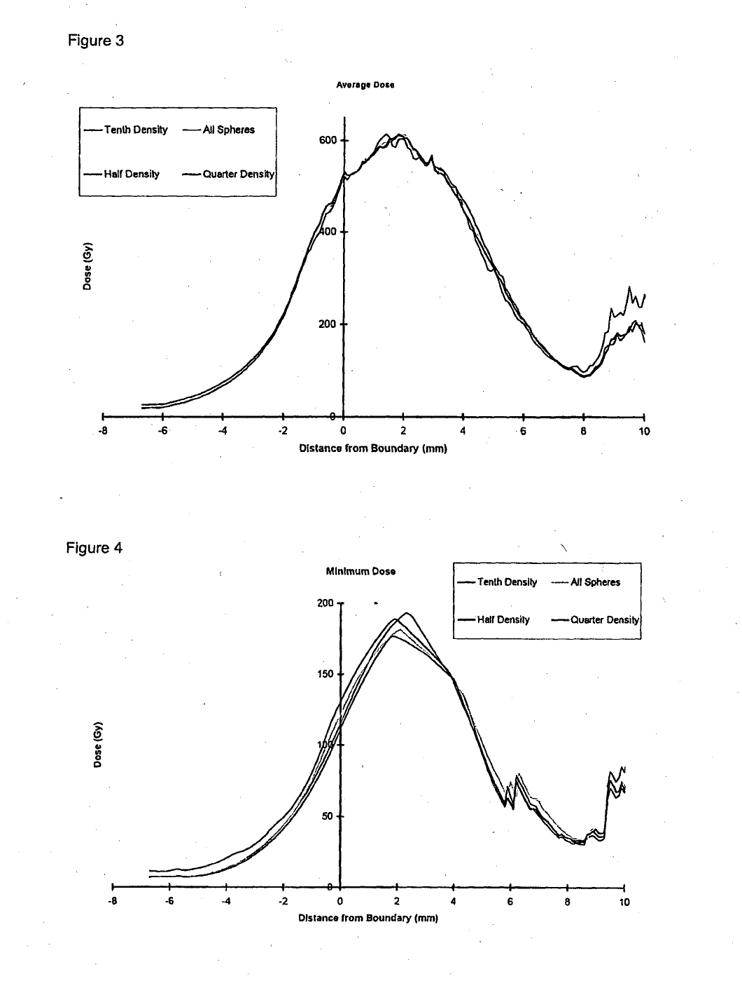

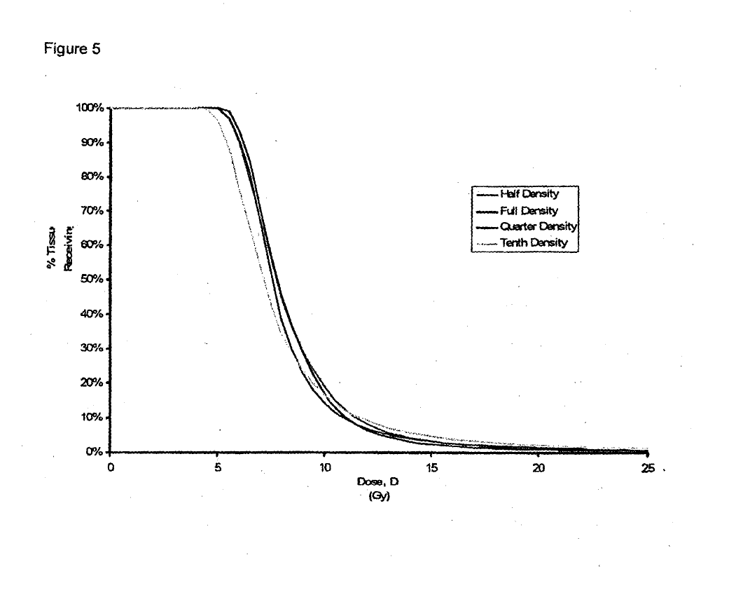

Similar to FIG. 1A, FIG. 2A shows selected isodose contours for a central tissue section through the tumour-normal tissue boundary of a liver tumour. However, the isodose contours have been modelled for microspheres of the present invention having a specific activity of 500 Bq per sphere when infused to deliver a 3 GBq dose (i.e. the total number of microspheres infused represents a tenth of the number of SIR-Spheres® required to deliver the equivalent radiation dose at 50 Bq per SIR-Sphere®). The “+” symbol indicates the position of radioactive microspheres having a specific activity of 50 Bq observed in tissue sections and the “0” indicates a tenth of those microspheres which were randomly selected to serve as the positions from which the isodose contours for 500 Bq microspheres have been determined. The dashed line on the left hand side shows the tumour boundary and the line on the right is the edge of the tumour vascular periphery, indicating that microsphere clusters predominantly occur in the tumour periphery.

FIG. 2B shows more detailed isodose contours of the boxed area shown in FIG. 2A.

FIG. 3 is a graph showing average radiation doses delivered across the tumour-normal tissue boundary for radioactive microspheres having a specific activity of 50 Bq (indicated by the curve labelled “All Spheres” and representing SIR-Spheres®), 100 Bq (indicated by the curve labelled “Half Density), 200 Bq (indicated by the curve labelled “Quarter Density”) and 500 Bq (indicated by the curve labelled “Tenth Density”). The tumour boundary is at 0 mm. Positive distances are inside the tumour.

FIG. 4 is a graph showing minimum radiation doses delivered across the tumour-normal tissue boundary for radioactive microspheres having a specific activity of 50 Bq (indicated by the curve labelled “All Spheres” and representing SIR-Spheres®), 100 Bq (indicated by the curve labelled “Half Density), 200 Bq (indicated by the curve labelled “Quarter Density”) and 500 Bq (indicated by the curve labelled “Tenth Density”). The tumour boundary is at 0 mm. Positive distances are inside the tumour.

FIG. 5 is a graph showing a cumulative dose-volume histogram for normal, non-target liver tissue from the tumour investigated and for which the results shown in FIGS. 1 to 4 were determined. The cumulative dose-volume curves for microspheres having a specific activity of 50 Bq (indicated by the curve labelled “Full Density” and representing SIR-Spheres®), 100 Bq (indicated by the curve labelled “Half Density), 200 Bq (indicated by the curve labelled “Quarter Density”) and 500 Bq (indicated by the curve labelled “Tenth Density”) are shown.

### DETAILED DESCRIPTION

Described herein are radioactive microspheres and methods for their production as well as methods and uses of these microspheres in Selective Internal Radiation Therapy (SIRT).

Selective Internal Radiation Therapy (SIRT) has long been practiced in the field of nuclear medicine to treat a range of cancers. SIRT has been applied very successfully as a treatment for liver cancers or tumours and the person skilled in the art would be well aware of methods perform SIRT. Notwithstanding, we note that descriptions of SIRT and associated procedures are publically available on several websites including the websites of Sirtex Medical Ltd. and Nordion, Inc. A/Prof Lourens Bester and Dr James Burnes provide a very useful description of SIRT using SIR-Spheres® on the website of the Royal Australian and New Zealand College of Radiologists (accessible at http://www.insideradiology.com.au/pages/view.php?T id=32#.UjfH0j HwZ1). In addition, the below listed publications authored by the present inventor describe SIRT:
Burton M of al. 1989 Selective Internal Radiation Therapy: Distribution of Radiation in the Liver. Eur. J Cancer Clin. Oncol. Vol 25. No 19. pp 1487;
Gray B N et al. 1992 Regression of Liver Metastases Following treatment with Yttrium-90 Microspheres. Aust. NZ. J Surgery. Vol 62. pp 105; and
Gray B N at al. 1989 Selective Internal Radiation (SIR) Therapy for treatment of Liver Metastases: Measurement of Response Rate. Vol 42 pp 192.

Similarly, the person skilled in the art would know how to manufacture radioactive microspheres based on what is by now common general knowledge in the field (Kawashita M at al. 1999, Preparation of phosphorus-containing silica glass microspheres for radiotherapy of cancer by ion implantation J Mater Sci Mater Med. August; 10(8):459-63; Conzone S D et al. 1998, Preparation and properties of radioactive rhenium glass microspheres intended for in vivo radioembolization therapy. J Biomed Mater Res. 1998 Dec. 15; 42(4):617-25; WO2002/34300 (US2003/0007928); E. L. R Hetherington 1999 Clinical development of holmium 166 microspheres for therapy of hepatic metastases. In IAEA-TECDOC-1114. Optimization of production and quality control of therapeutic radionuclides and radiopharmaceuticals. Final Report of a coordinated research project 1994-1998, Page 14-21).

A method of producing radioactive resin-based microspheres has been described in. WO2002/34300 and the microspheres of the present invention can be produced by following the basic method disclosed in WO2002/34300 but controlling the predetermined amount of radioactivity to be stably attached to the resin microspheres by adjusting the amount of resin throughout the method accordingly.

### Leach Test Method

A 5 μL sample of ⁹⁰Y labelled microspheres is diluted with water to 5 mL, adjusted to pH 7.0 and agitated in a water bath at 30° C. for 20 minutes.
A 100 μL sample is counted for beta emission in the Geiger-Müller counter. Another representative 100 μL sample is filtered through a 0.22 μm filter and the filtrate is counted for beta emission in the Geiger-Müller counter.
The percentage unbound ⁹⁰Y is calculated by:

$$\frac{Filtrate Count}{Sample Count} \times 100 = \% of unbound {}^{90} Y$$

As indicated above, the threshold amount of unbound (or unattached or unprecipitated) ⁹⁰Y in the production of these radioactive microspheres should be set at a maximum of 0.4%. If the leach test shows between 0.1-0.4% unbound ⁹⁰Y, then the microspheres are suitable for administration to patients.

The dosimetry of radioactive microspheres of the present invention has been investigated based on the dose distribution observed for microspheres having a specific activity of 50 Bq per microsphere. The radiation dose distribution of microspheres having a specific activity of 100 Bq, 200 Bq or 500 Bq was calculated and superimposed on previously reported dose distributions and calculated as described in the studies by Campbell et al. 2000 and 2001). The number of microspheres was reduced proportionally to the increase in specific activity. For example, only 1/10 of the number of 50 Bq microspheres was assessed when the specific activity of the microspheres was raised to 500 Bq (factor 10). Similarly, ½ of the number of 50 Bq microspheres was assessed when the specific activity of the microspheres was raised to 100 Bq (factor 2) and ¼ of the number of 50 Bq microspheres was assessed when the specific activity of the microspheres was raised to 200 Bq (factor 4).

The respective dose distributions were assessed for normal, non-target liver tissue as well as for tissue at the tumour-normal tissue boundary. In the calculation, allowance for smaller infused numbers of microspheres was made by randomly removing observed microsphere positions leaving either ½ or ¼ of the original number (see FIGS. 1 and 2). The removals were performed independently for each of the examples shown in FIG. 1 and FIG. 2, respectively.

Further, allowance for contributions to the overall radiation dose by beta emission from microspheres lying outside the sample was made after microsphere removal in accordance with what was previously described (Campbell et al. 2000). Briefly, where contributing microspheres were placed randomly in normal, non-target tissue or tissue towards the tumour centre they were placed at 50%, 25% or 10% of the observed tissue sample densities.

FIGS. 3 and 4 show average and minimum radiation doses delivered across the tumour-normal tissue boundary for radioactive microspheres having a specific activity of 50 Bq (indicated by the curve labelled “All Spheres” and representing SIR-Spheres®), 100 Bq (indicated by the curve labelled “Half Density), 200 Bq (indicated by the curve labelled “Quarter Density”) and 500 Bq (indicated by the curve labelled “Tenth Density”).

In light of the observations made by Vente et al. 2009 that infusion of a lower number of TheraSpheres® having a high specific activity appears to lead to undesirable dose distribution, potentially affecting the effectiveness of the TheraSpheres® in SIRT, it was surprisingly found that reducing the number of radioactive microspheres while proportionally increasing the specific activity per microsphere even by a factor of 10 did not have a significant effect on the radiation dose delivered and the calculated dose distribution suggests that the tumour periphery will receive a therapeutic dose for all of the three embodiments of the present invention tested.

It was also found that reducing the number of radioactive microspheres while proportionally increasing the specific activity per microsphere even by a factor of 10 did not have a significant effect on the radiation dose delivered to normal, non-target liver tissue. FIG. 5 shows the cumulative dose-volume histogram for normal, non-target liver tissue from the tumour investigated and for which the results shown in FIGS. 1 to 4 were determined. The cumulative dose-volume curves for microspheres having a specific activity of 50 Bq (indicated by the curve labelled “Full Density” and representing SIR-Spheres®), 100 Bq (indicated by the curve labelled “Half Density), 200 Bq (indicated by the curve labelled “Quarter Density”) and 500 Bq (indicated by the curve labelled “Tenth Density”) are shown.

In light of the above, it will be appreciated that raising the specific activity per microsphere in accordance with the present invention allows for a significant reduction in the number of microspheres to be infused to deliver a predetermined dose of radiation without compromising therapeutically effective distribution throughout the peripheral tumour vasculature.

The above also illustrates that the above-described undesirable side-effects seen in current SIRT practice can be minimised by using the microspheres of the present invention having an optimised specific radiation activity per microsphere and that the often serious clinical complications arising from current SIRT practice can be minimised without compromising the therapeutic effect.

Thus, while there has been described what are believed to be the preferred embodiments of the invention, those skilled in the art will recognize that other and further modifications may be made thereto without departing from the spirit of the invention, and it is intended to claim all such changes and modifications as falling within the scope of the invention. For example, any formulas given above are merely representative of procedures that, may be used. Steps may be added or deleted to methods described within the scope of the present invention.

1. A radioactive microsphere comprising a microsphere matrix and a radionuclide stably attached to, or incorporated within, said matrix,
wherein said microsphere has a diameter ranging from about 5 to about 45 μm, and
wherein said radionuclide provides a specific activity to said microsphere, said specific activity ranging from more than 100 Bq to less than 2000 Bq per microsphere.

2. The radioactive microsphere of claim 1, wherein said specific activity ranges from about 150 Bq to about 1500 Bq per microsphere, preferably from about 200 Bq to about 1500 Bq per microsphere, preferably from about 200 Bq to about 500 Bq per microsphere.

3. (canceled)

4. (canceled)

5. The radioactive microsphere of claim 1, wherein said specific activity ranges from more than 100 Bq to less than 1000 Bq per microsphere, preferably from 100 Bq to about 500 Bq per microsphere.

6. (canceled)

7. The radioactive microsphere of claim 1, for use in Selective Internal Radiation Therapy (SIRT).

8. The radioactive microsphere of claim 7, wherein said SIRT is SIRT to treat cancer.

9. The radioactive microsphere of claim 8, wherein said cancer is liver cancer or colorectal cancer.

10. (canceled)

11. The radioactive microsphere of claim 1, wherein said radionuclide is an isotope selected from isotopes of Yttrium, Holmium, Samarium, Iodine, Phosphorous, Iridium and Rhenium.

12. The radioactive microsphere of claim 11, wherein said radionuclide is an isotope of Yttrium, preferably 90-Yttrium (⁹⁰Y).

13. The radioactive microsphere of claim 1, wherein said microsphere matrix is a polymeric matrix.

14. The radioactive microsphere of claim 13, wherein said polymeric matrix is an ion exchange resin comprising partially cross-linked polystyrene, and
wherein said radionuclide is 90-Yttrium (⁹⁰Y) and wherein said ⁹⁰Y is precipitated as a ⁹⁰Y-phosphate salt such that said phosphate salt is stably attached to the surface of said ion exchange resin.

15. The radioactive microsphere of any one of claim 1, wherein said microsphere matrix is glass.

16. The radioactive microsphere of claim 1, wherein said microsphere provides for a radioactive dose distribution throughout a target tissue similar to that previously reported for polymeric microspheres having a specific activity of about 50 Bq per microsphere and significantly reduces the risk of undesirable side-effects of SIRT resulting from radioactive microsphere reflux.

17. A method of producing a radioactive microsphere comprising the step of combining a microsphere matrix with a radionuclide for a time and conditions sufficient to stably attach a specified portion of said radionuclide to said microsphere matrix, or to stably incorporate said specified portion of said radionuclide within said matrix, thereby producing a radioactive microsphere having a diameter ranging from about 5 to about 45 μm, wherein said specified portion of radionuclide provides a specific activity to said microsphere, said specific activity ranging from more than 100 Bq to less than 2000 Bq per microsphere.

18. A radioactive microsphere when produced by the method of claim 17.

19. A method of SIRT comprising administering the radioactive microsphere of claim 1 to a subject in need thereof.

20. (canceled)

21. The method of claim 19, wherein said subject has cancer and wherein said SIRT targets said cancer.

22. The method of claim 21, wherein said cancer is liver cancer or colorectal cancer.

23. Use of a radioactive microsphere of claim 1 for the manufacture of a medicament for the treatment of cancer.

24. (canceled)

25. The use of claim 23, wherein said medicament is adapted for use in SIRT.

26. The use of claim 25, wherein said SIRT targets said cancer, preferably, said cancer is liver or colorectal cancer.

27. (canceled)
